(19) United States
(12) Patent Application Publication
(10) Pub. No.: US 2005/0221408 A1
(43) Pub. Date: Oct. 6, 2005

(54) Compositions and methods for detection of single molecules

(75) Inventors: Eric A. Nalefski, Reading, MA (US); Steven R. Gullans, Natick, MA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: U.S. Genomics, Inc., Woburn, MA (US)

(21) Appl. No.: 11/087,095

(22) Filed: Mar. 21, 2005

Related U.S. Application Data
(60) Provisional application No. 60/554,792, filed on Mar. 19, 2004.
(60) Provisional application No. 60/555,484, filed on Mar. 22, 2004.
(60) Provisional application No. 60/566,646, filed on Apr. 30, 2004.

(51) Int. Cl. G01N 33/53; G01N 33/537; G01N 33/543

(52) U.S. Cl. 435/7.93

(57) ABSTRACT

The invention relates to compositions and methods for analyzing polymers such as proteins and their interactions with other molecules, including measuring affinity and kinetic constants.

### RELATED APPLICATIONS

This application claims priority to U.S. Provisional Application Ser. No. 60/554,792, entitled “SINGLE MOLECULE ANALYSIS OF BIOLOGICAL COMPONENTS”, filed Mar. 19, 2004, U.S. Provisional Application Ser. No. 60/555,484, entitled “METHODS FOR DETECTING AND QUANTIFYING MOLECULES IN A SAMPLE”, filed Mar. 22, 2004, and U.S. Provisional Application Ser. No. 60/566,646, entitled “SINGLE MOLECULE PROTEOMICS AND PHOSPHOPROTEOMICS”, filed Apr. 30, 2004, the entire contents of all of which are incorporated by reference herein.

### FIELD OF THE INVENTION

The invention relates to detection of single molecules such as proteins, particularly rare species thereof, and measurement of interactions involving such molecules.

### BACKGROUND OF THE INVENTION

The study of molecular and cellular biology is focused on the microscopic structure of cells. It is known that cells have a complex microstructure that controls the functionality of the cell. Much of the diversity associated with cellular structure and function is due to the ability of a cell to assemble various components into different cellular machinery. The cellular content of a cell is in turn governed in part by the transcriptional and translational control of the cell and by other interactions between cell components.

The ability to identify cellular components and the interactions each is capable of can be integral to the understanding of cellular function such as proliferation and differentiation.

There exists a need for more rapid and less laborious detection, measurement and analysis of molecules such as proteins and their interactions, particularly when such molecules are present at very low concentrations.

### SUMMARY OF THE INVENTION

The invention relates in part to analysis, including detection and measurement, of single molecules such as proteins. Modifications of such molecules can also be analyzed according to the invention. These modifications include but are not limited to post-translational modifications of proteins such as phosphorylation and glycosylation. These modifications may convert inactive proteins to active proteins (and vice versa) and thus the methods can also be used to assess active status of proteins. The invention also relates in part to analysis, including detection and measurement, of single nucleic acid molecules such as microRNA (miRNA).

The invention further relates in part to analysis, including detection and measurement, of complexes such as protein-containing complexes or nucleic acid-containing complexes. For example, the methods of the invention can detect interactions between proteins, between nucleic acids, between proteins and nucleic acids, between proteins with other components, and between nucleic acids with other components. These analyses can be performed at a single time point or at various times, thereby resulting in a time course. These analyses can also be performed in the presence or absence of other components, including for example candidate agonists or antagonists of such interactions. The invention provides methods for determining binding kinetics as well as binding affinities.

Thus, in one aspect, the invention provides a method for detecting a protein comprising contacting a sample with a first and a second protein-specific probe, and detecting the binding of both the first and the second protein-specific probe to a single protein as coincident signals. The first and the second protein-specific probes are labeled with first and second detectable labels, respectively, that are distinguishable from each other, and the binding of both the first and the second protein-specific probes to a single protein indicates that the protein is present in the sample. The first and second protein-specific probes are preferably different from each other and thus each recognizes and binds to the target protein in a manner different from other. For example, the first and second protein-specific probes may bind to different regions of the protein (e.g., different domains, different secondary structure, etc.).

In one embodiment, the first and second protein-specific probes are antibodies or antibody fragments, although they are not so limited.

In yet another embodiment, the method comprises contacting the sample with a third protein-specific probe that is labeled with a third detectable label, and detecting the binding of the first, second and third protein-specific probes to the protein as coincident signals.

In another embodiment, the first and second detectable labels are fluorophores. As an example, the first and second detectable labels are Alexa488 and Cy5, respectively. The third detectable label may be a fluorophore, but it is not so limited. As an example, the third detectable label may be Cy3.

In one embodiment, the second protein-specific probe and/or the third protein-specific probe may be specific for a protein modification such as but not limited to a phosphorylated amino acid residue.

The method may comprise detecting binding of the first and second protein-specific probes (or the first, second and third protein-specific probes) at a single time point or at various time points. The various time points may be equally or randomly spaced.

The method may further comprise detecting binding of the first and second protein-specific probes (or the first, second and third protein-specific probes) in the presence of another molecule. The other molecule may be a known molecule but it is not so limited. For example, the other molecule may be a candidate molecule that is being screened for its ability to modify the target protein or its ability to modulate modification of the protein by yet another molecule. In these latter embodiments, the method may comprise a screening method for identifying molecules with particular activities.

The method may further comprise comparing the level of binding of the first and second protein-specific probes to the level of binding of the first, second and third protein-specific probes.

The method may be used to detect more than one protein at a time. Thus, the method may further comprise detecting a second protein by contacting the sample with a second pair of probes specific for a second protein, each member of the second pair labeled with a distinguishable, detectable label. Similarly, the method can be used to detect a plurality of proteins and would thus comprise detecting a plurality of proteins by contacting the sample with pair of specific probes for each member of the plurality, wherein each member of a pair is labeled with a distinguishable, detectable label.

In one embodiment, the protein is present at a concentration of less than 1 ng/ml. In another embodiment, the protein is present at a concentration of below 30 fM. In yet another embodiment, the protein is present at a frequency of 1 in 2×10⁶ molecules in the sample.

In one embodiment, the sample is a blood, serum, plasma or urine sample. In another embodiment, the sample is a nanoliter volume.

In another embodiment, the first protein-specific probe is specific for a first chain and the second protein-specific probe is specific for a second chain in a quaternary structure comprising the protein. In yet another embodiment, the first and second protein-specific probes bind to an identical but repeating epitope on the protein.

In another aspect, the invention provides a method for detecting a microRNA (miRNA) comprising contacting a sample with a first and a second miRNA-specific probe, and detecting the binding of both the first and the second miRNA-specific probes to a single miRNA as coincident signals. The first and the second miRNA-specific probes are labeled with first and second detectable labels, respectively, that are distinguishable from each other, and the binding of both the first and the second miRNA-specific probes to a single miRNA indicates that the miRNA is present in the sample.

In one embodiment, the first and second miRNA-specific probes are nucleic acids. In another embodiment, the miRNA-specific probes are sequence-specific probes.

In one embodiment, the first and second detectable labels are fluorophores. The first and second detectable labels may be Alexa488 and Cy5, respectively, but they are not so limited provided they are distinguishable from each other.

In one embodiment, the method further comprises detecting a second miRNA by contacting the sample with a second pair of probes specific for the second miRNA, each labeled with a distinguishable detectable label. A plurality of miRNA may also be detecting by detecting a plurality of miRNA by contacting the sample with a plurality of probe pairs, each pair specific for a member of the miRNA plurality, and each member of each pair labeled with a distinguishable detectable label.

In one embodiment, the miRNA is present at a concentration of less than 1 ng/ml. In another embodiment, the miRNA is present at a concentration of below 30 fM. In yet another embodiment, the miRNA is present at a frequency of 1 in 2×10⁶ molecules in the sample. The sample may be a nanoliter volume.

In yet another aspect, the invention provides a method for detecting a complex comprising more than one component comprising contacting a sample with a first component-specific probe and a second component-specific probe, and detecting the binding of both the first component-specific probe and the second-component-specific probe to a single complex as coincident signals. The first component-specific probe and the second-component-specific probe are labeled with first and second detectable labels, respectively, that are distinguishable from each other, and the binding of both the first component-specific probe and the second-component-specific probe to a single complex indicates that the complex is present in the sample.

In one embodiment, the first component is a protein and the second component is a nucleic acid. In related embodiment, the first component-specific probe is an antibody or an antibody fragment and the second component-specific probe is a nucleic acid.

In another embodiment, the first component and the second component are both proteins. In a related embodiment, the first component-specific probe and the second-component specific probe are both antibodies or antibody fragments.

In yet another embodiment, the first component and second component are both nucleic acids, and in a related embodiment the first component-specific probe and the second component-specific probe are both nucleic acids.

In still another embodiment, the first component is an enzyme and the second component is a substrate. In yet another embodiment, the first component is a known molecule and the second component is putative binding partner of the first component.

In various embodiments, the first and second detectable labels are fluorophores. For example, the first and second detectable labels are Alexa488 and Cy5, respectively.

In one embodiment, the method may further comprise contacting the sample with a third component-specific probe that is labeled with a third detectable label and detecting binding of the first component-specific probe, the second component-specific probe and the third component-specific probe as coincident signals. The third detectable label may be a fluorophore, but it is not so limited.

In another embodiment, the method may further comprise contacting the sample with a plurality of component-specific probes, each of the plurality specific for separate component in the complex, and each labeled with a distinguishable, detectable label, and detecting binding of the plurality of component-specific probes as coincident signals.

In another aspect, the invention provides a method for detecting a complex comprising more than one component comprising contacting a first component-specific probe labeled with a first detectable label to a sample comprising a second component labeled with a second detectable label that is distinguishable from the first detectable label, and detecting binding of the first component-specific probe to a complex comprising the second component as coincident signals, wherein coincident signals indicate that the complex is present in the sample.

In one embodiment, the second component is intrinsically labeled. In another embodiment, the second component is labeled with a second component-specific probe that is labeled with the second detectable label.

Depending on the embodiment, the first component may be a protein and the second component may be a nucleic acid, or the first component and the second component may both be proteins, or the first component and second component may both be nucleic acids.

Depending on the embodiment, the first component-specific probe may be an antibody or an antibody fragment or a nucleic acid, and the second component-specific may be an antibody or antibody fragment or a nucleic acid. In other embodiments, the first component is an enzyme and the second component is a substrate. In still another embodiment, the first component is a known molecule and the second component is a putative binding partner of the first component, or vice versa.

In one embodiment, the first component and second component are labeled with distinguishable fluorophores, such as but not limited to Alexa488 and Cy5.

In yet another embodiment, the method further comprises contacting the sample with a third component-specific probe that is labeled with a third detectable label and detecting binding of the first component-specific probe and the third component-specific probe to the complex as coincident signals. The third detectable label may be a fluorophore but it is not so limited.

In another embodiment, the method further comprises contacting the sample with a plurality of component-specific probes, each of the plurality specific for a separate component in the complex, and each labeled with a distinguishable, detectable label, and detecting binding of the plurality of component-specific probes as coincident signals.

In yet another aspect, the invention provides a method for detecting a complex comprising more than one component comprising contacting a first component labeled with a first detectable label with a second component labeled with a second detectable label that is distinguishable from the first detectable label, and detecting binding of the first component to the second component as coincident signals, wherein coincident signals indicate that the complex is present.

In one embodiment, the first component is intrinsically labeled with a first detectable label. In another embodiment, the second component is intrinsically labeled with a second detectable label. In yet another embodiment, the first component is labeled with a first component-specific probe that is labeled with the first detectable label. In some embodiments, the second component is labeled with a second component-specific probe that is labeled with the second detectable label.

Depending on the embodiment, the first component and second component are both nucleic acids, or they are both proteins, or the first component is a protein and the second component is a nucleic acid.

In one embodiment, the first component-specific probe is an antibody or an antibody fragment. In another embodiment, the second component-specific probe is an antibody or an antibody fragment.

In another embodiment, the first component-specific probe is a nucleic acid. In still another embodiment, the second component-specific probe is a nucleic acid.

In still another embodiment, the first component is an enzyme and the second component is a substrate. In another embodiment, the first component is a known molecule and the second component is putative binding partner of the first component.

The first and second detectable labels may be fluorophores such as but not limited to Alexa488 and Cy5.

In one embodiment, the binding of the first component to the second component is measured in the presence of another molecule. The binding of the first component to the second component may be measured at a single time point or at various times.

In one embodiment, the method further comprises contacting a third component labeled with a third detectable label to the first component and the second component and detecting binding of the first component, second component and third component as coincident signals, wherein coincident signals indicate that a three component complex is present. The third detectable label may be a fluorophore, although it is not so limited.

The method may further comprise contacting a plurality of components each labeled with a distinguishable detectable label and detecting binding of one or more of the plurality as coincident signals.

In this and other aspects of the invention, the complex is present at a concentration of less than 1 ng/ml or below 30 fM. Similarly, the complex may also be present at a frequency of 1 in 2×10⁶ molecules in the sample. The sample may be a blood, serum, plasma or urine sample, but it is not so limited. The sample may be a nanoliter volume. These methods may be carried out using a single molecule detection or analysis system.

Each of the limitations of the invention can encompass various embodiments of the invention. It is therefore anticipated that each of the limitations of the invention involving any one element or combinations of elements can be included in each aspect of the invention. This invention is not limited in its application to the details of construction and/or the arrangement of components set forth in the following description or illustrated in the drawings. The invention is capable of other embodiments and of being practiced or of being carried out in various ways.

### BRIEF DESCRIPTION OF THE DRAWINGS

FIG. 1A is a schematic illustrating travel of molecules such as proteins or complexes in a sample flow past an interrogation spot. The proteins or complexes may be labeled with one or more probes as indicated by the presence of bound detectable labels. Unbound, detectably labeled probes also pass through the interrogation spot (or zone). Once in the interrogation spot, detectable labels such as fluorophores undergo laser excitation and their resultant emission is collected and measured. Dual labeled molecules produce simultaneous emission whereas free (i.e., unbound) probes emit a single color.

FIG. 1B is a representative screen capture showing the emission profile of a sample analyzed according to FIG. 1A. The screen capture contains 50 milliseconds of data. It contains numerous dual color coincident peaks (indicated by arrows).

FIG. 1C is a graph showing the results of a probe titration experiment to determine the range of sensitivity of the assay. A linear relationship between the concentration of a dual labeled oligonucleotide and the number of coincident peaks detected is observed over several orders of magnitude, with sensitivity in the low- to mid-femtomolar range (inset) and low inter-run variability.

FIG. 2A is a schematic illustrating the binding of detectably and distinguishably labeled antibody probes (Ab) to an antigen (Ag) (e.g., a protein). An antibody probe pair is designed such that each member of the pair recognizes and binds to a separate region of the antigen, thereby ensuring maximal coincident (e.g., concurrent or simultaneous) binding of the pair to the antigen.

FIG. 2B is a schematic illustrating the emission profile of a sample of unbound, detectably and distinguishably labeled antibody probes. Each probe is individually detected, since the probability that two probes will co-exist in the interrogation spot is small. The dotted line represents a set threshold.

FIG. 2C is a schematic illustrating the emission profile of a sample containing unbound, detectably and distinguishably labeled antibody probes and a dually labeled antigen (e.g., a protein). The unbound probes are detected as individual, temporally-separated peaks while the antigen is detected by two overlapping peaks of the distinguishable, detectable labels. The dotted line represents a set threshold.

FIG. 3 is a graph illustrating the results of an assay for IL-6 using a dual colored, coincident detection approach. Individual molecules of IL-6 were detected as coincident blue-red (e.g., Alexa488-Cy5) using polyclonal antibodies individually labeled with Alexa488 and Cy5. Results are represented as the average plus standard deviation (bars) of three determinations. The number of molecules is linearly dependent on IL-6 concentration. The sensitivity of the assay is less than 1 ng/ml.

FIG. 4A is a schematic illustrating the domain structure of Akt1 and the role of phosphorylation on its activation. PH represents the plekstrin homology domain. Cat represents the catalytic domain. Reg represents the regulatory domain.

FIG. 4B is a schematic illustrating the use of three antibody probes to distinguish between phosphorylated (i.e., active) and non-phosphorylated (i.e., inactive) Akt1. Two antibody probes (e.g., PH-specific and C-terminal domain-specific antibodies) are used to detect active and inactive Akt1 and another antibody is used to detect the phosphorylated amino acid residue (i.e., pSer473). Active Akt1 can be accomplished with one Akt1 antibody probe and one pSer473 antibody probe or with two Akt2 antibody probes and one pSer473 antibody probe. Each of the antibody probes used is distinguishably labeled.

FIG. 4C is a schematic illustrating the antibody probe binding to an unphosphorylated (i.e., inactive) Akt1. This approach can be used to detect Akt1 regardless of its active or inactive state.

FIG. 4D is a schematic illustrating the antibody probe binding to a phosphorylated (i.e., active) Akt1. Active Akt1 is detected as a blue-green-red coincident peak whereas total Akt1 is detected as a green-red coincident peak. The use of a three color system distinguishes between active Akt1 and inactive Akt1. If one is interested in detecting only active Akt1 without knowledge of total Akt1 or conversion to inactive Akt1, then the approach can be simplified to one antibody probe to Akt1 (either to the PH-domain or the Reg domain, for example) and one antibody to pSer473.

FIG. 5A is a bar graph showing the number of two color coincident peaks detected in the presence of active Akt1, in the presence of inactive Akt1, and in the presence of a non-Akt1 control (i.e., GST) when using an antibody to the PH domain and an antibody to the C-terminus of Akt1.

FIG. 5B is a bar graph showing the number of two color coincident peaks detected in the presence of active Akt1, in the presence of inactive Akt1, and in the presence of a non-Akt1 control (i.e., GST) when using an antibody to the PH domain and an antibody to pSer473.

FIG. 5C is a graph showing the linear relationship between percent of active Akt1 in a sample and the number of three color coincident peaks when using an antibody to the PH domain, and antibody to the C-terminus, and one antibody to pSer473 of Akt1. Each sample contained 100 nM total Akt1 comprised of the various fractions of active Akt1 with the remainder being inactive Akt1. The number of molecules detected increases linearly with the proportion of active Akt1 molecules due to a fractional increase in the number of enzyme molecules phosphorylated on Ser473.

FIG. 6A is a schematic showing interaction between a protein and a nucleic acid molecule. The protein may be a transcription factor that binds a nucleic acid. In the figure, a nucleic acid binding domain that is a zinc finger domain (ZFD) is shown.

FIG. 6B is a schematic showing the emission profile of a sample containing non-interacting but distinguishably labeled protein (e.g., a ZFD) and nucleic acid to which the protein can bind. In the absence of interaction between the protein and the nucleic acid, each is detected as an individual peak.

FIG. 6C is a schematic showing the emission profile of a sample containing non-interacting nucleic acid and protein, each of which is detected as an individual peak, and a nucleic acid-protein complex, which is detected as a pair of overlapping peaks. If the interaction between the nucleic acid and protein is reversible, a time course analysis can be performed to determine on and off rates of binding. In another approach, binding affinities of components can be determined by measuring amounts of bound and unbound components at equilibrium. In yet another approach, proteins that bind to a particular nucleic acid (or nucleotide sequence) may be detected and optionally isolated. It is to be understood that this analysis can be performed for any multicomponent system for which probes specific to each component are available, or where it is possible to generate components that are inherently labeled, for example, during synthesis.

FIG. 7A is a graph showing the number of coincident peaks observed as a function of free target DNA concentration at equilibrium. The coincident peaks represent binding of a ZFD to the target DNA. Free target DNA concentration is determined using single Cy5 peaks. Kd is a function of the protein-DNA complexes relative to free DNA. The number of coincident peaks was corrected for random coincidence of the molecules in the interrogation spot. Results are presented as the mean plus the standard deviation (bars) of three determinations. The solid line represents best-fitting to a single-site binding equation. The inset represents the same data presented on a semi-log plot.

FIG. 7B is a graph showing the kinetics of ZFD binding to its target DNA. Dissociation kinetics of protein-DNA complexes were measured after pre-binding the ZFD to its Cy5-labeled target DNA and then initiating dissociation by addition of 100-fold molar excess unlabeled target DNA. The inset shows association kinetics of ZFD-DNA complexes measured after mixing free ZFD with Cy5-labeled target DNA. Solid lines represent best-fitting to mono-exponential equations.

FIG. 8A is a schematic showing an intensity versus time profile for a sample that lacks target antigen. The target antigen is detected using antibodies that recognize an epitope that is repeated on the antigen. Each intensity peak corresponds to an unbound antibody.

FIG. 8B is a schematic showing an intensity versus time profile for a sample containing antigen (Ag) when that antigen is detected using antibodies that recognize an epitope that is repeated on the antigen. Binding of at least two, and preferably more, antibodies to an antigen results in an intensity signal that is greater than the signal of a single bound antibody. The aggregation of antibodies onto a single antigen therefore leads to a greater signal and can be used to identify peaks that correspond to antigen rather than random noise in the system.

FIGS. 9A and 9B are representative 200 millisecond screenshots showing raw data in the absence (9A) and presence (9B) of antigen. In this example, antibody and antigen final concentrations were 70 pM and 10 pM respectively.

FIG. 10 is a graph showing the shift in intensity peak heights that can occur when protein aggregation is used to distinguish antigen signal from random noise.

FIG. 11 is a bar graph showing the quantitation of immune complexes in the presence or absence of antigen.

The Figures are illustrative only and are not required for enablement of the invention.

### DETAILED DESCRIPTION OF THE INVENTION

The invention provides methods for single molecule detection. The invention is capable of detecting single molecules including single proteins, single nucleic acids (e.g., miRNA or siRNA), and single complexes potentially comprising either or both of the foregoing. It is further able to analyze changes in such single molecules including phosphorylation and dephosphorylation events, and association or dissociation events. In doing so, it provides methods for determining the status of a single molecule, such as active or inactive status of a protein, methods for determining affinity and binding constants of a molecule for another molecule, such as binding affinity of a protein for another protein or nucleic acid, reaction kinetics thereof, and the like. It is also capable of use in a screening method for identifying and isolating factors that interfere or promote any of the above phenomena.

The methods provided herein involve the ability to detect single molecules or single complexes based on the temporally coincident detection of detectable labels specific to the proteins being analyzed or the individual components of the complexes being analyzed. As used herein, coincident detection refers to the detection of an emission signal from more than one detectable label in a given period of time. Generally, the period of time is short, approximating the period of time necessary to analyze a single molecule. As shown in the Figures and Examples, this time period may be on the order of a millisecond. Also as shown in the Figures, coincident detection is manifest as emission signals that overlap as a function of time. The co-existence of the emission signals in a given time frame may indicate that two non-interacting molecules, each individually and distinguishably labeled, are present in the interrogation spot at the same time. An example would be the simultaneous presence of two unbound but detectably and distinguishably labeled probes in the interrogation spot. However, because the spot volume is so small (and the corresponding analysis time is so short), the likelihood of this happening is small. Rather it is more likely that if two probes are present in the interrogation spot simultaneously, this is due to the binding of both probes to a single molecule passing through the spot.

The coincident detection methods of the invention involve the simultaneous detection of more than one emission signal. The number of emission signals that are coincident will depend on the number of distinguishable detectable labels available, the number of probes available, the number of components being detected, the nature of the detection system being used, etc. Generally, at least two emission signals are being detected. In some embodiments, three emission signals are being detected. However, the invention is not so limited. Thus, where multiple components are being detected in a single analysis, 4, 5, 6, 7, 8, 9, 10, 25, 50, 100, 500, 1000 or more emission signals can be detected simultaneously.

Coincident binding refers to the binding of two or more probes on a single molecule or complex. Coincident binding of two or more probes is used as an indicator of the molecule or complex of interest. Coincident binding may take many forms including but not limited to a color coincident event, whereby two colors corresponding to a first and a second detectable label are detected. Coincident binding may also be manifest as the proximal binding of a first detectable label that is a FRET donor fluorophore and a second detectable label that is a FRET acceptor fluorophore. In this latter embodiment, a positive signal is a signal from the FRET acceptor fluorophore upon laser excitation of the FRET donor fluorophore.

Coincident detection analysis is able to detect single molecules at very low concentrations. For example, as discussed herein, low femtomolar concentrations of proteins and other single molecules can be detected using a two or three emission signal approach. In addition, the analysis demonstrates a dynamic range of greater than four orders of magnitude. A two emission signal approach is also able to detect single molecules such as single proteins at levels below 1 ng/ml.

As used herein, a protein is a polymer made up of amino acid monomers (or residues) linked together by peptide bonds. A protein therefore includes a peptide. A protein also includes fragments or regions (e.g., domains) of naturally or non-naturally occurring proteins. Amino acid monomers may be naturally or non-naturally occurring amino acids. Proteins to be detected are referred to herein as “target” proteins.

Depending on the application, the protein may be intrinsically labeled with a detectable label (e.g., during synthesis using a detectably labeled amino acid). A molecule that is intrinsically labeled does not require a separate probe in order to be detected. Rather the intrinsic label is sufficient for rendering the molecule detectable. Alternatively, and more commonly, the protein is labeled by binding to it a specific probe (i.e., it is extrinsically labeled). The probe may be a sequence- or structure-specific probe, wherein the sequence or structure recognized and bound by the probe is sufficiently unique to that protein.

Accordingly, the probe may recognize a primary, secondary or tertiary structure of a target protein. A primary structure of a protein is a linear arrangement of amino acids. A secondary structure of a protein refers to the folding of the peptide “backbone” chain into various conformations that may result in distant amino acids being brought into proximity with each other. Examples of secondary structure include alpha helices, beta pleated sheets, or random coils. A tertiary structure of a protein is its overall three dimensional structure. A quaternary structure of a protein is the structure formed by its noncovalent interaction with one or more other macromolecules (such as other proteins). An example of a quaternary structure is the structure formed by the four globin protein subunits to make hemoglobin. The probes of the invention may be specific for any of the afore-mentioned structures. A quaternary structure may also be recognized as a complex, as described herein.

The probe may itself be a protein but it is not so limited. Examples of suitable protein-specific probes include antibodies and antibody fragments, nucleic acids (e.g., aptamers that recognize protein targets), protein substrates (preferably non-catalyzable), and the like. Antibodies include polyclonal and monoclonal antibodies and further include IgG, IgA, IgM, IgE, IgD as well as antibody variants such as single chain antibodies. Antibody fragments contain at least an antigen-binding site and thus include but are not limited to Fab and F(ab)₂ fragments.

The methods provided herein involve the use of probes that bind to the target molecule in a specific manner. Specific binding as used herein means the probe binds to the target with greater affinity than it does to other molecules. The probe may bind to other molecules, but preferably such binding is at or near background levels. The affinity of the probe for the protein of interest may be at least 2-fold, at least 5-fold, at least 10-fold, or more than its affinity for other molecules. Probes with the greatest differential affinity are preferred in most embodiments, although they may not be those with the greatest affinity for the target.

Single proteins are therefore generally detected using at least two probes that are specific to the protein (i.e., protein-specific probes, as discussed herein). Two probes are generally sufficient although a greater number may be used depending on the application. A sample may be tested for the presence of a protein by contacting it with two or more protein-specific probes for a time and under conditions that allow for binding of the probes to the protein if it is present. Excess amounts of both probes may be used to ensure that all binding sites are occupied. The probes are preferably chosen so that they bind to different regions of the protein, and therefore cannot compete with each other for binding to the protein. The probes are also labeled with distinguishable detectable labels (i.e., the detectable label on the first probe is distinct from that on the second probe). Once the probes are allowed to bind to the protein (if it is present in the sample), the sample is analyzed for coincidence emission signals. For example, a protein bound by both probes is manifest as overlapping emission signals from the bound probes. This can be accomplished using a single molecule detection or analysis system. A single molecule detection or analysis system is a system capable of detecting and analyzing individual molecules.

The method is particularly suited to detecting proteins in a rare or small sample (e.g., a nanoliter volume sample) or in a sample where protein concentration is low. The invention allows more than one and preferably several different proteins to be detected simultaneously, thereby conserving sample. In other words, the method is capable of a high degree of multiplexing. For example, the degree of multiplexing may be 2 (i.e., 2 proteins can be detected in a single analysis), 3, 4, 5, 6, 7, 8, 9, 10, at least 20, at least 50, at least 100, at least 500, or higher. Each protein is detected using a particular probe pair where each member of the probe pair is specific to the protein and each probe used in an analysis is labeled with a distinguishable label. Thus, a plurality of proteins may be detected and analyzed. As used herein, a plurality is an amount greater than two but less than infinity. A plurality is sometimes less than a million, less than a thousand or less than a hundred.

The Examples demonstrate the ability to detect protein levels using sandwich immunoassays using antibodies as the protein-specific probes. Two antibodies are used, each of which is labeled with a fluorophore. This assay is sensitive to less than 1 ng/mL antigen, a level that is sufficient to detect rare-abundance protein markers in plasma samples, for instance. The Examples demonstrate detection of interleukin 6 (IL-6) using polyclonal antibodies. Polyclonal antibodies can be used in this and other aspects of the invention since it should be possible to generate polyclonal antibodies to virtually any molecule. Polyclonal antibodies are a heterogeneous mixture of antibodies directed against a particular molecule. Each antibody in the mixture may recognize a different epitope on the target molecule.

The Examples also demonstrate detection of Akt1. Akt1 has three domains, a plekstrin homology (PH) domain, a catalytic (Cat) domain, and a regulatory (Reg) domain. Antibodies to the PH and Reg domains were used to detect Akt1.

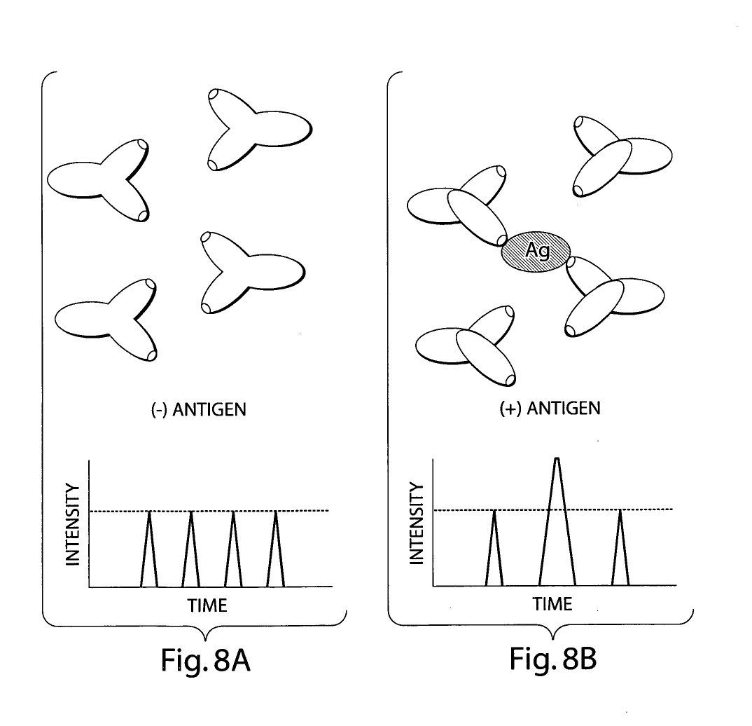

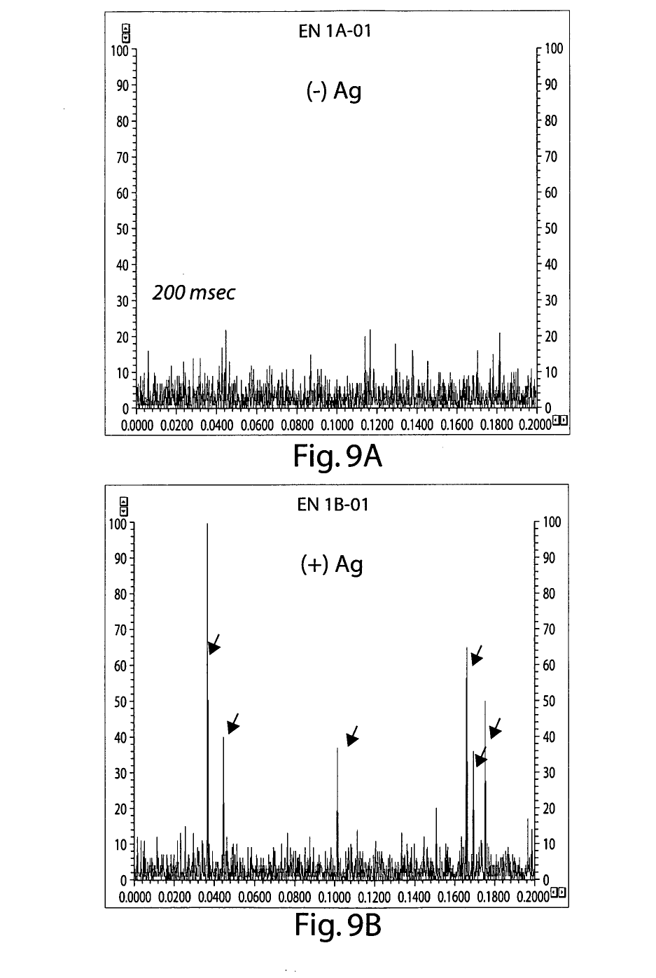

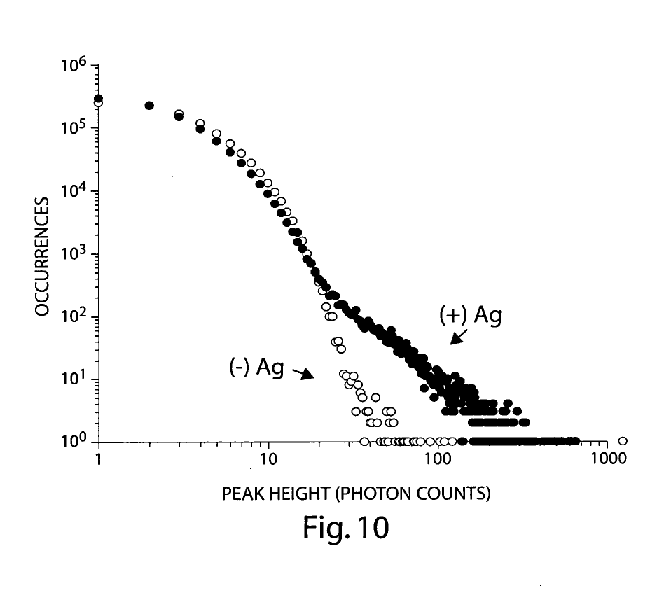

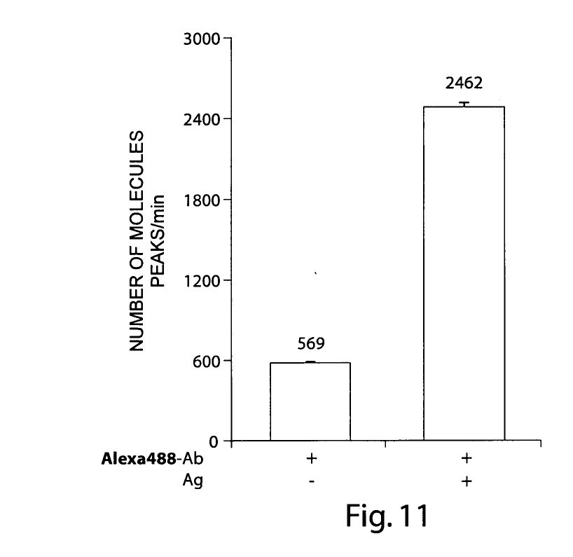

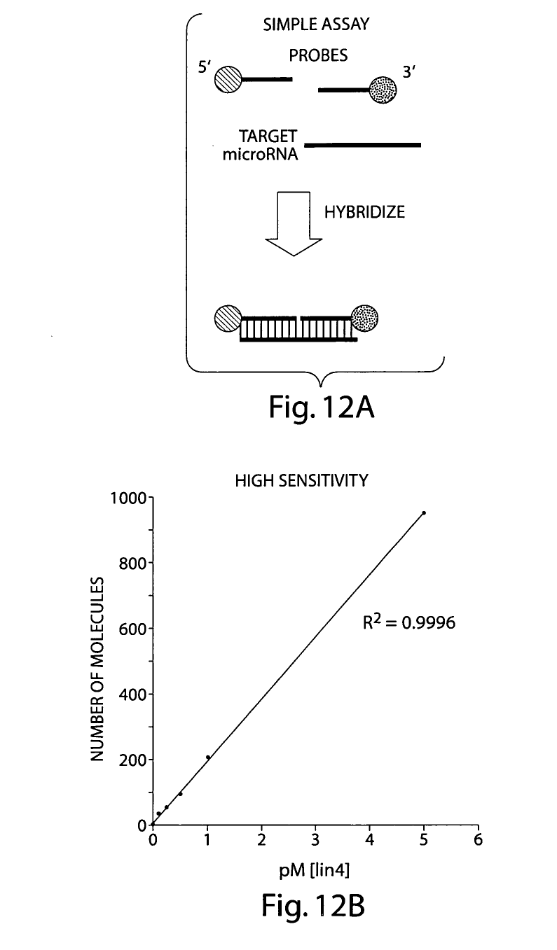

In another aspect of the invention, the target is detected using a probe that is capable of binding to the target molecule at multiple locations, thereby allowing for signal amplification and distinction from random noise in the system. As shown in FIGS. 8-11, it is possible to detect the presence of a target protein or nucleic acid via the aggregation of a plurality of probes such as antibodies (or fragments thereof). If the ratio of target to probe is one, there is less probability that the antigen will be detected. However, by increasing the number of probes bound to a given target molecule, there is a greater probability of detecting the target due to the increased total signal emitted by the complex.

The invention is not limited to the proteins that can be detected, provided that specific probes are available for the protein of interest. Proteins that can be detected include those of clinical significance such as those usually detected in a clinical sample such as urine or blood (including plasma and serum). These include growth factors and cytokines, hormones, tissue leakage proteins, and classical plasma proteins used in standard diagnosis protocols such as those listed in Harrison's Principles of Experimental Medicine, 13^{th} Edition, McGraw-Hill, Inc., N.Y. Proteins to be detected also include those having forensic value such as HLA markers and antibodies directed against them, ABO blood types and antibodies directed against them, phosphoglucomutase (PGM 2-1), erythrocyte acid phosphatase (EAP), esterase D (EsD), adenyl kinase (AK), adenosine deaminase (ADA), glutamic pyruvate transaminase (GPT), 6-phosphogluconate dehydrogenase (6-PGD), glucose-6-phosphate dehydrogenase (G-6-PD) and transferrin (Tf). Virtually any protein having a polymorphism that can be detected with a specific probe can be a target in the forensic methods of the invention. Other proteins to be detected include signal transduction proteins such as receptor kinases, adaptor molecules, etc., transcription factors, developmental mediators, histone and histone modifying enzymes such as deacetylases, phosphatases, lipid modifying enzymes, and the like.

It is also possible to measure the presence, absence and level of a protein as a function of time or conditions, as described herein.

The invention can be used to determine the relative concentration or absolute amount of a protein in a sample. The relative concentration or amount is determined by measuring the amount of coincident signal from probes coincidentally bound to a protein. The coincident signal level can be compared to a standard calibration curve that is prepared prior to or at the same time as the test solution is analyzed for absolute quantitation. The standard calibration curve is a plot of signal intensity (y-axis) as a function of known protein concentration (x-axis). Those of ordinary skill will be familiar with the generation of such curves. A similar approach can be used to determine the concentration of other molecules, such as but not limited to miRNA or siRNA.

The probes may be specific for any region of the protein of interest. In some embodiments, the probe is specific for a protein modification. Protein modifications include post-translational modifications such as phosphorylation, glycosylation, ubiquitinylation, acetylation, and the like. The only limitation on the type of modification that can be detected is the availability of specific probes for each.

In the case of phosphorylation, a probe may recognize a phosphorylated residue on a particular protein. If the protein exists in a phosphorylated and dephosphorylated form, then the probe (when used alone) will recognize only a subset of the protein. However, by combining the phosphorylation specific probe with other probes that recognize other regions of the protein, it is possible to determine the percentage of protein that is phosphorylated. It is further possible to study the rate of conversion to a phosphorylated or dephosphorylated form by performing this analysis as a time course.

The Examples illustrate the ability to detect protein phosphorylation using Akt1 as an example. The Ser/Thr kinase Akt1 is phosphorylated at residue Ser473. Antibodies are available that recognize and bind specifically to Ser473 when it is phosphorylated (i.e., pSer473). The phosphorylated form of Akt1 is active whereas the dephosphorylated form is inactive. In these Examples, the phosphorylated form is detected using one antibody probe to the PH domain, one antibody probe to the C-terminal (or Reg) domain, and one antibody to pSer473. Coincidence detection of red, green and blue signals is indicative of the phosphorylated and thus active form of Akt1. Binding of the pSer473-specific antibody does not interfere with binding of the C-terminal antibody. Therefore, total Akt1 is represented by the total number of red-green coincidences since both active and inactive species of Akt1 will bind the PH and C-terminal antibodies regardless of whether the protein is phosphorylated.

Single molecule detection offers a rapid and quantitative method for the discovery and characterization of biological modifiers such as inhibitors and targets of kinases and phosphatases. In the Akt1 Example, the presence of phosphorylated Akt1 can be determined in the presence, absence, or changing concentration of one or more other molecules. It is to be understood that these screening assays are not limited solely to effects on Akt1. Rather they can be adapted to any protein capable of being phosphorylated, provided a suitably specific probe is available.

Antibodies specific for various modifications can be obtained from commercial sources such as Sigma, Pharmingen and the like or they can be synthesized by techniques known in the art.

The methods of the invention can also be used to analyze complex presence, formation, dissociation, and content. As used herein, a complex is two or more components bound to each other ionically or non-ionically. As used herein, each member in a complex is called a component. The components may be the same or different from each other. The components may be proteins, nucleic acids, chemical compounds, and the like. Examples of complexes include protein-protein complexes, protein-nucleic acid complexes, nucleic acid-nucleic acid complexes, enzyme-substrate complexes, and the like. A complex may have 2, 3, 4, 5, 6, 7, 8, 9, 10, or more components.

Complexes are detected using a probe specific to each component of the complex, and detecting coincident signals from such probes. Thus, a two component complex is detected by coincident signals from two probes, one for each component. Similarly, a three component complex is detected by coincident signals from three probes, one for each component. The probes used for this purpose are referred to as component-specific probes. Thus, a probe that recognizes a first component of a complex is referred to as a first component-specific probe. Similarly, a probe that recognizes a second component of a complex is referred to as a second component-specific probe.

Depending on the embodiment, the components may be intrinsically labeled with a detectable label, thereby avoiding the need for an additional probe to detect that particular component. For example, if the complex being analyzed is a protein-nucleic acid complex, then it is possible to use a nucleic acid that has a detectable label incorporated into its structure during its synthesis. Similarly, a protein may intrinsically comprise a detectable label using detectably labeled amino acid residues for its synthesis. These components may be more appropriate when analyzing the effects of other molecules on complex formation (e.g., screening of complex inhibitors).

The Examples demonstrate detection of complexes of transcription factors and their nucleic acid targets. In these Examples, the nucleic acid is double stranded and has been labeled with a Cy5 fluorophore. The protein is the zinc finger nucleic acid binding domain of Early Growth Response Protein Egr-1 and it has been labeled with BODIPY-F1. Analysis of formation and dissociation via the appearance and disappearance of temporally coincident Cy5 and BODIPY signals as a function of time yields information regarding association rates, dissociation rates, and therefore affinities of the components for each other, and ultimately stability of the complex. The data analysis used in such methods is standard in the art.

As mentioned herein, it is possible to study the effect of other molecules on complex formation and stability. These other compounds include putative agonists or antagonists of complex formation. Such compounds may be derived from libraries containing naturally and/or non-naturally occurring compounds. The invention provides a sensitive method to detect the effect of each of these library members on the complex of interest.

The invention can also be used to identify compounds with particular affinities to a known component. In these embodiments, one component of a complex is known and the aim is to identify and isolate compounds that bind to that component. The latter compounds are referred to herein as “binding partners” of the known component. The putative binding partners may be in a library such as that described above. In this embodiment, however, the library members may be detectably labeled (with the same label) prior to their contact with the known component. The known compound is labeled with a detectable label that is distinct from other labels used in the same assay. Coincident signals from the detectably labeled known component and a library member indicates that the library member has a particular binding affinity for the known component. The degree of affinity can be analyzed further by modulating the environment such as increasing or decreasing salt concentration, pH, and temperature, for example.

The term “nucleic acid” is used herein to mean multiple nucleotides (i.e., molecules comprising a sugar (e.g., ribose or deoxyribose) linked to an exchangeable organic base, which is either a substituted pyrimidine (e.g., cytosine (C), thymidine (T) or uracil (U)) or a substituted purine (e.g., adenine (A) or guanine (G)). As used herein, the terms refer to oligoribonucleotides as well as oligodeoxyribonucleotides. The terms shall also include polynucleosides (i.e., a polynucleotide minus a phosphate) and any other organic base containing polymer. Nucleic acids can be obtained from existing nucleic acid sources (e.g., genomic or cDNA), or by synthetic means (e.g., produced by nucleic acid synthesis).

“Sequence-specific” probes when used in the context of a nucleic acid means that the probe recognizes a particular linear (or quasi-linear) arrangement of nucleotides or derivatives thereof. In preferred embodiments, the probe is itself composed of nucleic acid elements such as DNA, RNA, PNA and LNA elements and combinations thereof (as discussed below). In preferred embodiments, the linear arrangement includes contiguous nucleotides or derivatives thereof that each binds to a corresponding complementary nucleotide in the probe. In some embodiments, however, the sequence may not be contiguous as there may be one, two, or more nucleotides that do not have corresponding complementary residues on the probe.

Any molecule that is capable of recognizing a nucleic acid with structural or sequence specificity can be used. In most instances, such probes will form at least a Watson-Crick bond with the nucleic acid target. In other instances, the nucleic acid probe can form a Hoogsteen bond with the nucleic acid target, thereby forming a triplex. A nucleic acid probe that binds by Hoogsteen binding enters the major groove of a nucleic acid target and hybridizes with the bases located there. Examples of these latter probes include molecules that recognize and bind to the minor and major grooves of nucleic acids (e.g., some forms of antibiotics). In some embodiments, the nucleic acid probes can form both Watson-Crick and Hoogsteen bonds with the nucleic acid target. Bis PNA probes, for instance, are capable of both Watson-Crick and Hoogsteen binding to a nucleic acid.

The length of nucleic acid probe can also determine the specificity of binding. The energetic cost of a single mismatch between the probe and the nucleic acid target is relatively higher for shorter sequences than for longer ones. Therefore, hybridization of smaller nucleic acid probes is more specific than is hybridization of longer nucleic acid probes because the longer probes can embrace mismatches and still continue to bind to the nucleic acid depending on the conditions. One potential limitation to the use of shorter probes however is their inherently lower stability at a given temperature and salt concentration. In order to avoid this latter limitation, bis PNA probes can be used to bind shorter sequences with sufficient hybrid stability.

Notwithstanding these provisos, nucleic acid probes can be any length ranging from at least 4 nucleotides to in excess of 1000 nucleotides. In preferred embodiments, the probes are 5-100 nucleotides in length, more preferably between 5-25 nucleotides in length, and even more preferably 5-12 nucleotides in length. The length of the probe can be any length of nucleotides between and including the ranges listed herein, as if each and every length was explicitly recited herein. Thus, the length may be at least 5 nucleotides, at least 10 nucleotides, at least 15 nucleotides, at least 20 nucleotides, or at least 25 nucleotides, or more, in length. It should be understood that not all residues of the probe need hybridize to complementary residues in the nucleic acid target. For example, the probe may be 50 residues in length, yet only 25 of those residues hybridize to the nucleic acid target. Preferably, the residues that hybridize are contiguous with each other.

The nucleic acid probes are preferably single stranded, but they are not so limited. For example, when the probe is a bis PNA it can adopt a secondary structure with the nucleic acid target resulting in a triple helix conformation, with one region of the bis PNA clamp forming Hoogsteen bonds with the backbone of the polymer and another region of the bis PNA clamp forming Watson-Crick bonds with the nucleotide bases of the target.

The nucleic acid probe hybridizes to a complementary sequence within the nucleic acid target. The specificity of binding can be manipulated based on the hybridization conditions. For example, salt concentration and temperature can be modulated in order to vary the range of sequences recognized by the nucleic acid probes. Those of ordinary skill in the art will be able to determine optimum conditions for a desired specificity.

The nucleic acid target may be labeled in a sequence non-specific manner in addition to the sequence-specific labeling discussed herein. For example, a DNA backbone may be stained with a backbone label. Examples of backbone stains that label nucleic acids in a sequence non-specific manner include intercalating dyes such as phenanthridines and acridines (e.g., ethidium bromide, propidium iodide, hexidium iodide, dihydroethidium, ethidium homodimer-1 and -2, ethidium monoazide, and ACMA); minor grove binders such as indoles and imidazoles (e.g., Hoechst 33258, Hoechst 33342, Hoechst 34580 and DAPI); and miscellaneous nucleic acid stains such as acridine orange (also capable of intercalating), 7-AAD, actinomycin D, LDS751, and hydroxystilbamidine. All of the aforementioned nucleic acid stains are commercially available from suppliers such as Molecular Probes, Inc.

Still other examples of nucleic acid stains include the following dyes from Molecular Probes: cyanine dyes such as SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red).

Additionally, the nucleic acid target can be synthesized in a manner that incorporates fluorophores directly into the growing nucleic acid. For example, this latter labeling can be accomplished by chemical means or by the introduction of active amino or thiol groups into nucleic acids. (Proudnikov and Mirabekov, Nucleic Acid Research, 24:4535-4532, 1996.) An extensive description of modification procedures that can be performed on a nucleic acid polymer can be found in Hermanson, G. T., Bioconjugate Techniques, Academic Press, Inc., San Diego, 1996, which is incorporated by reference herein.

There are several known methods of direct chemical labeling of DNA (Hermanson, 1996; Roget et al., 1989; Proudnikov and Mirabekov, 1996). One of the methods is based on the introduction of aldehyde groups by partial depurination of DNA. Fluorescent labels with an attached hydrazine group are efficiently coupled with the aldehyde groups and the hydrazine bonds are stabilized by reduction with sodium labeling efficiencies around 60%. The reaction of cytosine with bisulfite in the presence of an excess of an amine fluorophore leads to transamination at the N4 position (Hermanson, 1996). Reaction conditions such as pH, amine fluorophore concentration, and incubation time and temperature affect the yield of products formed. At high concentrations of the amine fluorophore (3M), transamination can approach 100% (Draper and Gold, 1980).

In addition to the above method, it is also possible to synthesize nucleic acids de novo (e.g., using automated nucleic acid synthesizers) using fluorescently labeled nucleotides. Such nucleotides are commercially available from suppliers such as Amersham Pharmacia Biotech, Molecular Probes, and New England Nuclear/Perkin Elmer.

The invention is also suited to the detection of nucleic acid molecules, such as microRNA and siRNA, both of which are commonly present at low levels. miRNA and siRNA are relatively short RNA molecules ranging in length from about 7-35 nucleotides. They are able to interfere with translation from mRNA species and can therefore control protein expression in a cell.

The invention contemplates measurement of these RNA species using two or more probes. The probes should not interfere with each other's binding to the RNA and so preferably will recognize and bind to different regions of the target RNA. Suitable probes may be sequence-specific probes that recognize and bind to a linear arrangement of nucleotides in their target, usually in a complementary manner.

In some embodiments, the probe is a peptide nucleic acid (PNA), a bis PNA clamp, a locked nucleic acid (LNA), a ssPNA, a pseudocomplementary PNA (pcPNA), a two-armed PNA, DNA, RNA, or co-nucleic acids of the above such as DNA-LNA co-nucleic acids (as described in co-pending U.S. Patent Application having Ser. No. 10/421,644 and publication number U.S. 2003-0215864 A1 and published Nov. 20, 2003, and PCT application having serial number PCT/US03/12480 and publication number WO 03/091455 A1 and published Nov. 6, 2003, filed on Apr. 23, 2003), or co-polymers thereof (e.g., a DNA-LNA co-polymer).

FIG. 12A is a schematic of an exemplary assay for nucleic acids such as miRNA and siRNA (e.g., synthetic duplex 21-mers). Differentially labeled probes are used, each of which binds (e.g., hybridizes if the probe is a nucleic acid also) to a different region of the target miRNA. Detection of both detectable labels on one molecule is indicative of the presence of that particular target. Hybridization conditions may be varied according to the level of stringency and specificity required in the assay. FIG. 12B shows the results of an analysis for lin-4 miRNA spiked into a total RNA background. The assay shows sub-picomolar sensitivity, high specificity and broad dynamic range. It has also been possible to measure changes in miRNA levels in a temporally dependent manner (e.g., during development). In a C. elegans model system, it was possible to measure lin-4 levels during and through the L1, L2, L3 and adult stages with results that correlated with those previously reported (data not shown).

Using the methods described herein, it has also been possible to detect changes in mRNA levels following siRNA exposure. The methods of the invention yielded results that correlated with RT-PCR results (data not shown).

The methods and system provided herein demonstrate a sensitivity on the order of 3 fM (approximating 1 copy per 100 million total background molecules) and a dynamic range of 4+logs and a CV less than 10%.

The samples to be tested can be biological or bodily samples such as tissue biopsies, urine, sputum, semen, stool, saliva and the like. The sample in some instances can be analyzed as is without harvest and/or isolation of the molecules of interest. Alternatively, harvest and isolation of proteins, nucleic acids or complexes can be performed and methods for doing so are routinely practiced in the art and can be found in standard molecular biology textbooks (e.g., such as Maniatis' Handbook of Molecular Biology).

In important embodiments, the sample has a nanoliter volume. That is, it is only necessary to load a nanoliter volume into the detection system in order to perform the method described herein. In still other important embodiments, the protein is present at a frequency of 1 in 1,000,000 molecules or 1 in 2,000,000 molecules in the sample. Accordingly, the method can be used to detect and analyze proteins that are extremely rare.

Although the proteins and nucleic acids may be linearized or stretched prior to analysis, this is not necessary if the detection system is capable of analyzing both stretched and condensed versions. This is particularly the case when coincident events are being detected since these events simply require the presence or absence of at least two labels, but are not necessarily dependent upon the relative positioning of the labels (provided however that if they are being detected using FRET, they are sufficiently proximal to each other to enable energy transfer between each other). In some instances, it may not even be desirable to modify the conformation of the protein or complex particularly if the probe is one that recognizes a secondary, tertiary or quaternary structure.

As used herein, stretching of the target protein or nucleic acid means that it is provided in a substantially linear extended (e.g., denatured) form rather than a compacted, coiled and/or folded (e.g., secondary) form. Stretching the protein prior to analysis may be accomplished using particular configurations of, for example, a single molecule detection system, in order to maintain the linear form. These configurations are not required if the target can be analyzed in a compacted form.

The sample or reaction mixture may be cleaned prior to analysis. As used herein “cleaning” refers to the process of removing unbound probes. This cleaning step can be accomplished in a number of ways including but not limited to column purification. Column purification generally involves capture of small molecules within a column with flow-through of larger molecules (such as the target proteins). It is to be understood however that the method can be performed without removal of these reagents prior to analysis, particularly since coincident detection can distinguish between desired binding events and artifacts. Thus, in some embodiments, the unbound detectable probes are not removed prior to analysis.

The targets and probes of the invention are detectably labeled, either intrinsically or extrinsically. A detectable label is a moiety, the presence of which can be ascertained directly or indirectly. Generally, detection of the label involves the creation of a detectable signal such as for example an emission of energy. The label may be of a chemical, peptide or nucleic acid nature although it is not so limited. The nature of label used will depend on a variety of factors, including the nature of the analysis being conducted, the type of the energy source and detector used and the type of target. The label should be sterically and chemically compatible with the constituents to which it is bound. Detectable labels may be, for example, light emitting, energy accepting, fluorescing, radioactive, quenching, and the like, as the invention is not limited in this respect. Guidelines for selecting the appropriate labels, and methods for adding extrinsic labels to polymers are provided in more detail in U.S. Pat. No. 6,355,420 B1.

The detectable label can be directly or indirectly detected. A directly detectable moiety is one that can be detected directly by its ability to emit and/or absorb light of a particular wavelength. An indirectly detectable moiety is one that can be detected indirectly by its ability to bind, recruit and, in some cases, cleave another moiety which may in turn emit or absorb light of a particular wavelength. An example of indirect detection is the use of a first enzyme label which cleaves a substrate into directly detectable products. The label may be organic or inorganic in nature. For example, it may be chemical, peptide or nucleic acid in nature although it is not so limited. Labels can be conjugated to a polymer or probe using thiol, amino or carboxylic groups.

More specifically, the detectable label may be selected from the group consisting of directly detectable labels such as a fluorescent molecule (e.g., fluorescein, rhodamine, tetramethylrhodamine, R-phycoerythrin, Cy-3, Cy-5, Cy-7, Texas Red, Phar-Red, allophycocyanin (APC), fluorescein amine, eosin, dansyl, umbelliferone, 5-carboxyfluorescein (FAM), 2′7′-dimethoxy-4′5′-dichloro-6-carboxyfluorescein (JOE), 6 carboxyrhodamine (R6G), N,N,N′,N′-tetramethyl-6-carboxyrhodamine (TAMRA), 6-carboxy-X-rhodamine (ROX), 4-(4′-dimethylaminophenylazo)benzoic acid (DABCYL), 5-(2′-aminoethyl)aminonaphthalene-1-sulfonic acid (EDANS), 4-acetamido-4′-isothiocyanatostilbene-2,2′disulfonic acid, acridine, acridine isothiocyanate, r-amino-N-(3-vinylsulfonyl)phenylnaphthalimide-3,5, disulfonate (Lucifer Yellow VS), N-(4-anilino-1-naphthyl)maleimide, anthranilamide, Brilliant Yellow, coumarin, 7-amino-4-methylcoumarin, 7-amino-4-trifluoromethylcouluarin (Coumarin 151), cyanosine, 4′, 6-diaminidino-2-phenylindole (DAPI), 5′,5″-diaminidino-2-phenylindole (DAPI), 5′,5″-dibromopyrogallol-sulfonephthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2,2′-disulfonic acid, 4,4′-diisothiocyanatostilbene-2,2′-disulfonic acid, 4-dimethylaminophenylazophenyl-4′-isothiocyanate (DABITC), eosin isothiocyanate, erythrosin B, erythrosin isothiocyanate, ethidium, 5-(4,6-dichlorotriazin-2-yl) aminofluorescein (DTAF), QFITC (XRITC), fluorescamine, IR144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene, pyrene butyrate, succinimidyl 1-pyrene butyrate, Reactive Red 4 (Cibacron™ Brilliant Red 3B-A), lissamine rhodamine B sulfonyl chloride, rhodamine B, rhodamine 123, rhodamine X, sulforhodamine B, sulforhodamine 101, sulfonyl chloride derivative of sulforhodamine 101, tetramethyl rhodamine, riboflavin, rosolic acid, and terbium chelate derivatives), a chemiluminescent molecule, a bioluminescent molecule, a chromogenic molecule, a radioisotope (e.g., P³² or H³, ¹⁴C, ¹²⁵I and ¹³¹I), an electron spin resonance molecule (such as for example nitroxyl radicals), an optical or electron density molecule, an electrical charge transducing or transferring molecule, an electromagnetic molecule such as a magnetic or paramagnetic bead or particle, a semiconductor nanocrystal or nanoparticle (such as quantum dots described for example in U.S. Pat. No. 6,207,392 and commercially available from Quantum Dot Corporation and Evident Technologies), a colloidal metal, a colloid gold nanocrystal, a nuclear magnetic resonance molecule, and the like.

The detectable label can also be selected from the group consisting of indirectly detectable labels such as an enzyme (e.g., alkaline phosphatase, horseradish peroxidase, β-galactosidase, glucoamylase, lysozyme, luciferases such as firefly luciferase and bacterial luciferase (U.S. Pat. No. 4,737,456); saccharide oxidases such as glucose oxidase, galactose oxidase, and glucose-6-phosphate dehydrogenase; heterocyclic oxidases such as uricase and xanthine oxidase coupled to an enzyme that uses hydrogen peroxide to oxidize a dye precursor such as HRP, lactoperoxidase, or microperoxidase), an enzyme substrate, an affinity molecule, a ligand, a receptor, a biotin molecule, an avidin molecule, a streptavidin molecule, an antigen (e.g., epitope tags such as the FLAG or HA epitope), a hapten (e.g., biotin, pyridoxal, digoxigenin fluorescein and dinitrophenol), an antibody, an antibody fragment, a microbead, and the like.

The label may additionally be selected from the group consisting of an electron spin resonance molecule (such as for example nitroxyl radicals), a fluorescent molecule (i.e., fluorophores), a chemiluminescent molecule (e.g., chemiluminescent substrates), a radioisotope, an optical or electron density marker, an enzyme, an enzyme substrate, a biotin molecule, a streptavidin molecule, an electrical charge transferring molecule (i.e., an electrical charge transducing molecule), a chromogenic substrate, a semiconductor nanocrystal, a semiconductor nanoparticle, a colloid gold nanocrystal, a ligand, a microbead, a magnetic bead, a paramagnetic particle, a quantum dot, an affinity molecule, a protein, a peptide, nucleic acid, a carbohydrate, an antigen, a hapten, an antibody, an antibody fragment, and a lipid.

The nature of the detection system used will depend upon the nature of the detectable labels used. The detection system can be selected from any number of detection systems known in the art. These include an electron spin resonance (ESR) detection system, a charge coupled device (CCD) detection system, an avalanche photodiode (APD) detection system, a photomultiplier (PMT) detection system, a fluorescent detection system, an electrical detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) detection system, many of which are electromagnetic detection systems.

Detection of bound antibodies is accomplished by techniques known to those skilled in the art. Antibodies may be detected by either directly labeling them with a detectable label or by binding to them a secondary antibody that is itself detectably labeled and that is specific for the primary antibody bound to the target.

In some embodiments, the detectable labels all emit distinguishable signals that are detectable using one type of detection system. For example, the detectable moieties can all be fluorescent labels. In other embodiments, the detectable labels can be detected using different detection systems. For example, one probe may be labeled with a fluorophore while another may be labeled with a radioisotope.

In some instances, the detectable labels are part of a FRET system with fluorescence signals dependent upon the proximal location of donor and acceptor molecules.

As used herein, “conjugated” means two entities stably bound to one another by any physicochemical means. It is important that the nature of the attachment is such that it does not substantially impair the effectiveness of either entity. Keeping these parameters in mind, any covalent or non-covalent linkage known to those of ordinary skill in the art is contemplated unless explicitly stated otherwise herein. Non-covalent conjugation includes hydrophobic interactions, ionic interactions, high affinity interactions such as biotin-avidin and biotin-streptavidin complexation and other affinity interactions. Such means and methods of attachment are known to those of ordinary skill in the art. Conjugation can be performed using standard techniques common to those of ordinary skill in the art. For example, U.S. Pat. Nos. 3,940,475 and 3,645,090 demonstrate conjugation of fluorophores and enzymes to antibodies.

For instance, functional groups which are reactive with various labels include, but are not limited to, (functional group: reactive group of light emissive compound) activated ester:amines or anilines; acyl azide:amines or anilines; acyl halide:amines, anilines, alcohols or phenols; acyl nitrile:alcohols or phenols; aldehyde:amines or anilines; alkyl halide:amines, anilines, alcohols, phenols or thiols; alkyl sulfonate:thiols, alcohols or phenols; anhydride:alcohols, phenols, amines or anilines; aryl halide:thiols; aziridine:thiols or thioethers; carboxylic acid:amines, anilines, alcohols or alkyl halides; diazoalkane:carboxylic acids; epoxide:thiols; haloacetamide:thiols; halotriazine:amines, anilines or phenols; hydrazine:aldehydes or ketones; hydroxyamine:aldehydes or ketones; imido ester:amines or anilines; isocyanate:amines or anilines; and isothiocyanate:amines or anilines.

Polymers may be analyzed using a single molecule analysis system. A single molecule analysis system is capable of analyzing single molecules separately from other molecules. Such a system is sufficiently sensitive to detect signals emitting from a single molecule and its bound probes. The system may be a linear molecule analysis system in which single molecules are analyzed in a linear manner (i.e., starting at a point along the polymer length and then moving progressively in one direction or another). In certain embodiments in which detection is based predominately on the presence or absence of a signal such as a coincident signal, linear analysis may not be required. The system may be a direct molecule analysis system in which single polymers are analyzed in their totality with multiple probes and labels are detected simultaneously.

The system is preferably not an electrophoretic method and thus is sometimes referred to as a non-electrophoretic single molecule detection (or analysis) system. Such systems do not rely on gel electrophoresis or capillary electrophoresis to separate molecules from each other.

An example of a single molecule detection/analysis system is the Trilogy™ instrument which is based on the Gene Engine™ technology described in PCT patent applications WO98/35012 and WO00/09757, published on Aug. 13, 1998, and Feb. 24, 2000, respectively, and in issued U.S. Pat. No. 6,355,420 B1, issued Mar. 12, 2002. This latter system allows single polymers to be passed through an interaction station, whereby the units of the polymer or labels of the compound are interrogated individually in order to determine whether there is a detectable label conjugated to the polymer/compound/complex. Interrogation involves exposing the label to an energy source such as optical radiation of a set wavelength. In response to the energy source exposure, the detectable label emits a detectable signal. The mechanism for signal emission and detection will depend on the type of label sought to be detected.

The Trilogy™ technology does not require linear analysis of molecules and rather analyzes molecules in their totality. The Trilogy™ provides real-time counting of individually labeled molecules in a nanoliter interrogation zone. The system detects labeled molecules at low femtomolar concentrations and displays a dynamic range over 4+logs. The system can accommodate standard sample carriers such as but not limited to 96 well plates or microcentrifuge (e.g., Eppendorf) tubes. The sample volumes may be on the order of microliters (e.g., 1 ul volume).

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1

### Single-Molecule Detection Experimental Platform

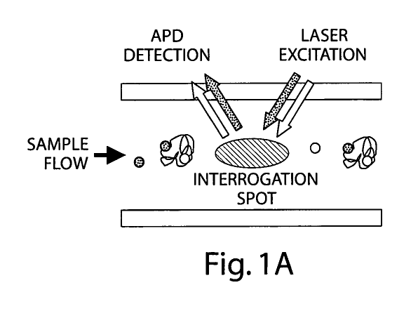

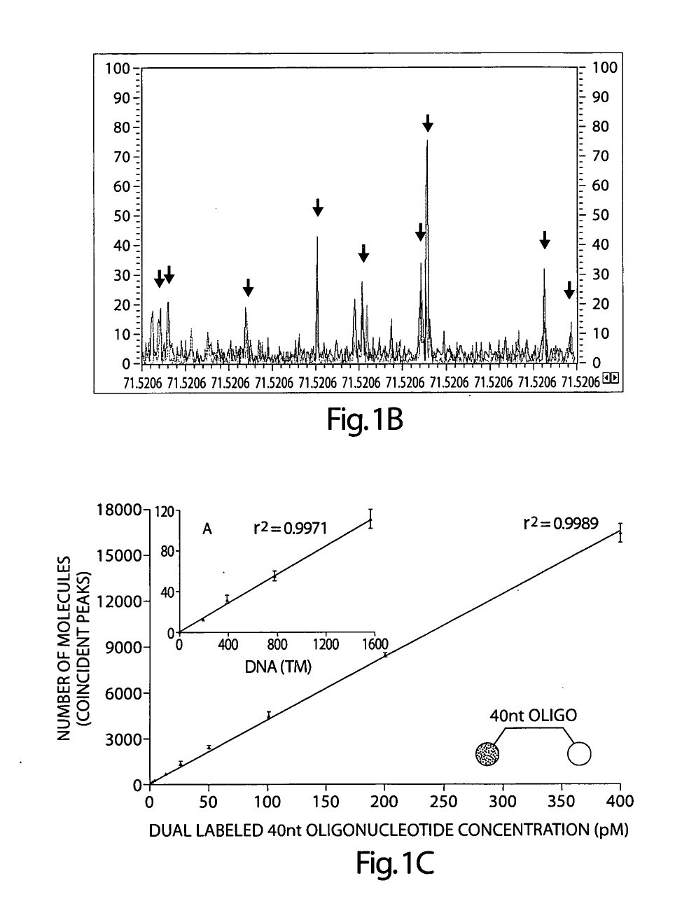

Single molecule detection may be accomplished using a U.S. Genomics Trilogy™ instrument. The U.S. Genomics Trilogy™ instrument comprises integrated confocal optics, multi-color laser interrogation and detection, microfluidics and on-board software for instrument control, data capture and analysis. A schematic diagram of a sample containing fluorescently labeled molecules (DNA, RNA or protein) as it flows through the instrument is presented in FIG. 1A. The sample moves through the microfluidic chambers where fluorophores undergo laser excitation and the resulting emission is measured. Dual-labeled molecules produce simultaneous emission of two colors, while free fluorophores or unbound probes emit a single color. In FIG. 1B, a 50 ms screenshot identifying numerous red-green coincident peaks as detected is shown. Titration of a dual-labeled DNA oligo showing linear response over several orders of magnitude, with sensitivity in the lower femtomolar range (inset) and low inter-run variability is presented in FIG. 1C.

### Example 2

### Single-Molecule Immunoassay

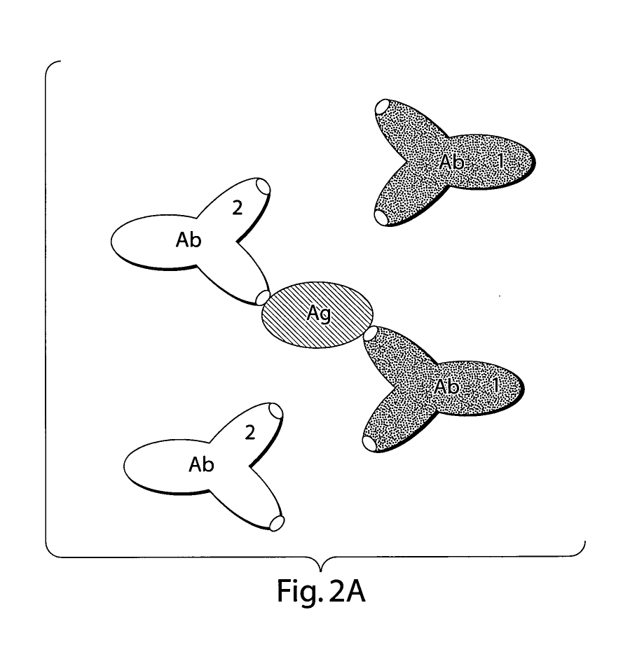

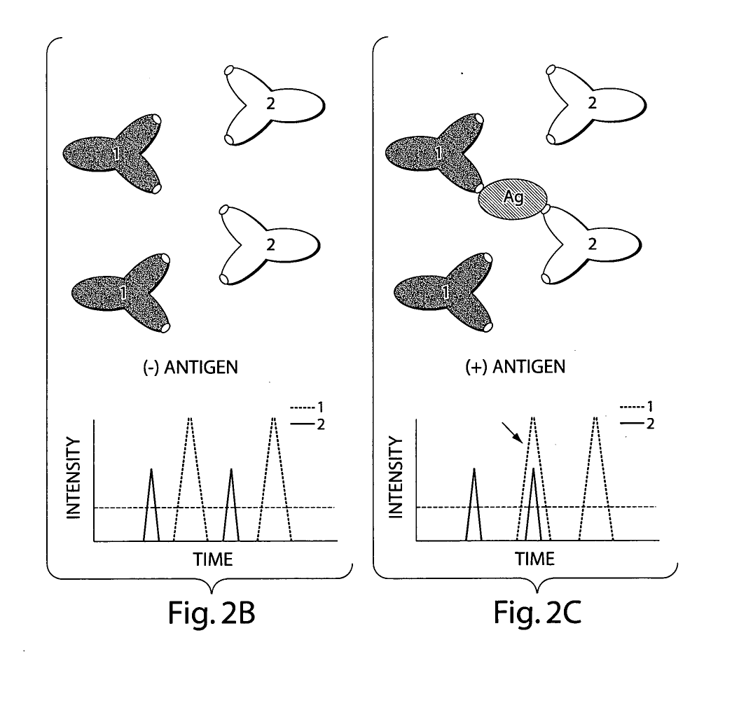

Formation of immune complexes composed of an antigen (Ag) sandwiched between multiple antibodies (Ab) is depicted in the schematic diagram shown in FIG. 2A. FIGS. 2B and 2C include a graph depicting data from the single molecule immunoassay as fluorescence intensity versus time. In the absence of antigen (FIG. 2B), individual dye-labeled antibody molecules pass through the detection zone independently, and each appears as a discrete fluorescence peak above a set threshold. In the presence of antigen (FIG. 2C), a pair of differently colored dye-labeled antibodies bound to antigen pass simultaneously through the detection zone, and the immune complex appears as a pair of coincident peaks.

### Example 3

### Quantitation of Protein Levels Using Single Molecule Immunoassays

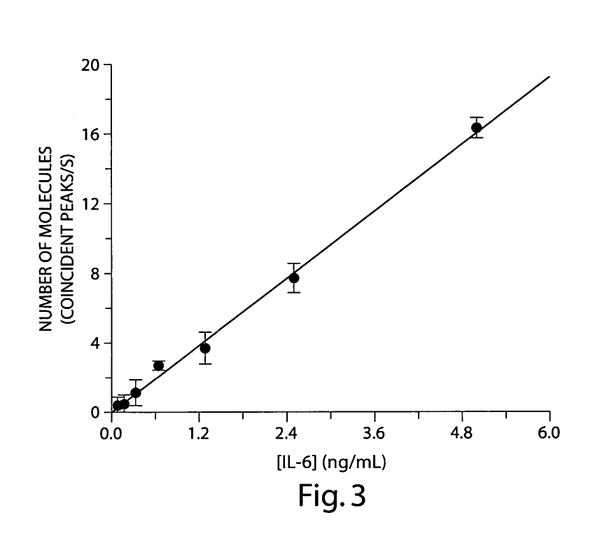

Protein levels were quantitated using single molecule immunoassays and the U.S. Genomics Trilogy™ instrument. Individual molecules of IL-6 were detected as coincident Blue-Red (Alexa488-Cy5) peaks in solutions containing IL-6 and a mixture of polyclonal antibodies labeled separately with Alexa488 and Cy5. Results presented in FIG. 3 are presented as the average plus standard deviation (bars) of three determinations. The number of molecules detected is linearly dependent on IL-6 concentration. The sensitivity of this particular assay is <1 ng/mL.

### Example 4

### Quantitation of Specific Phosphorylation States of Akt1 Using Single-Molecule Immunoassays

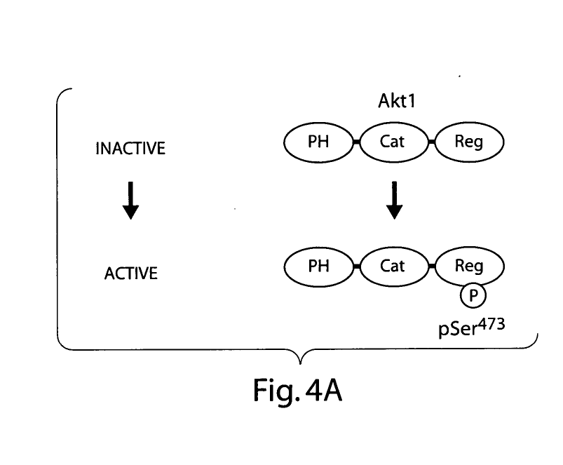

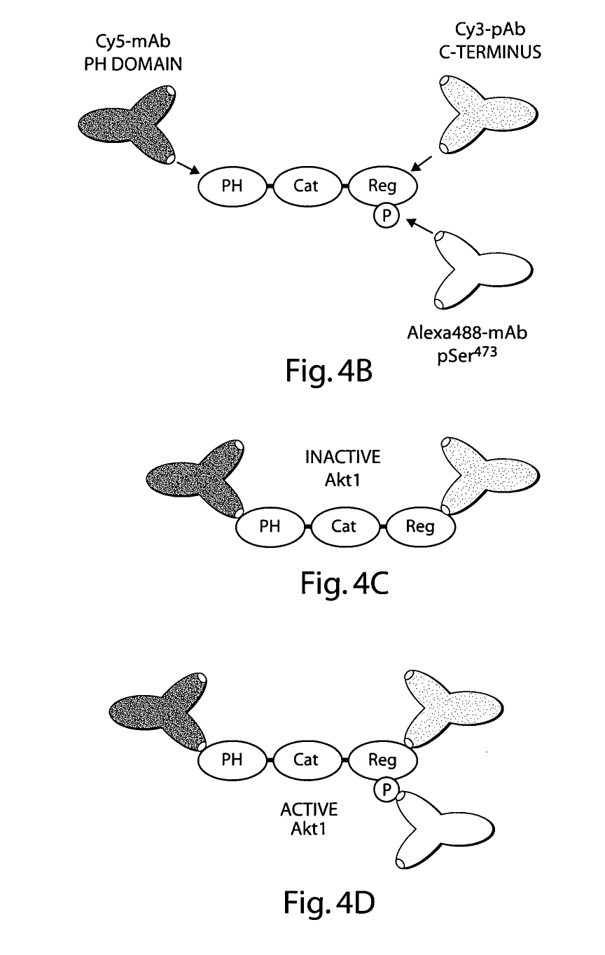

A schematic of Ser-/Thr-kinase Akt1 and its activation by phosphorylation is presented in FIG. 4A. Akt1 (i.e., PKB) plays a central role in cellular responses such as transcription, protein synthesis, glycogen synthesis, cell growth and survival, and angiogenesis. Brazil and Hemmings 2001, TIBS 26:657. Akt1 is converted from an inactive to active enzyme by phosphorylation of the regulatory domain residue Ser473, as shown in FIG. 4A.

Fluorophore labeled antibodies specific for activation states of Akt1 are depicted in FIG. 4B. FIG. 4C shows the binding of PH domain-specific monoclonal antibodies (labeled with Cy5 (red)) and C-terminus-specific polyclonal antibody (labeled with Cy3 (green)) to Akt1. FIG. 4D shows binding of a PH domain-specific monoclonal antibody labeled with Cy5 (red), a C-terminus-specific polyclonal antibody labeled with Cy3 (green), and an Akt1 phospho-Ser473-specific monoclonal antibody labeled with Alexa488 (blue). After reacting a mixture containing all three antibodies with Akt1, both inactive and active Akt1 molecules are detected as green-red (Cy3-Cy5) coincident peaks, whereas only active Akt1 molecules are detected as blue-green-red (Alexa488-Cy3-Cy5) coincident peaks.

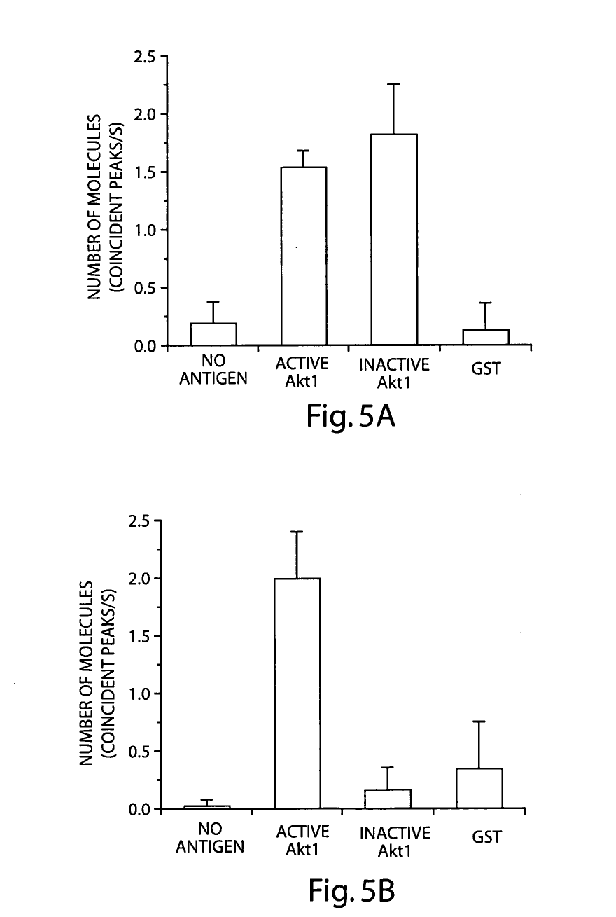

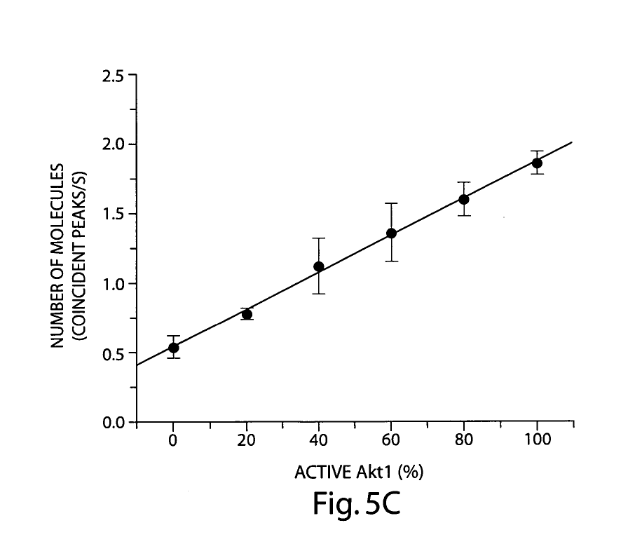

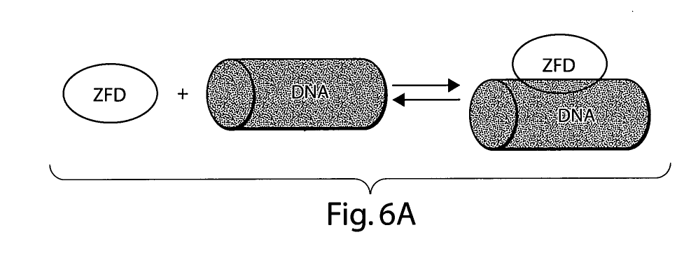

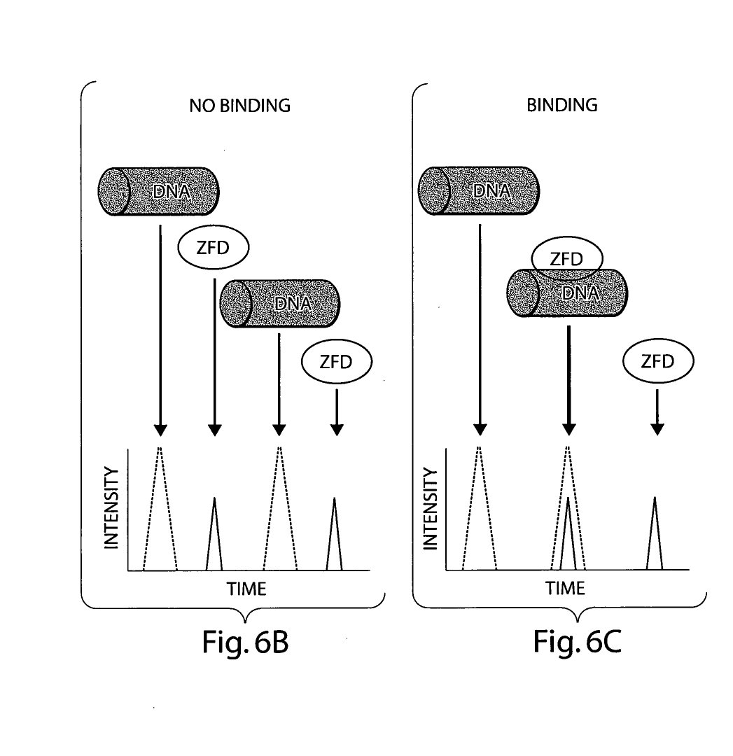

FIG. 6 shows the data obtained to quantitate specific phosphorylation states of Akt1 using single-molecule immunoassays. Akt1 was reacted with a mixture of three differently colored antibodies, and each sample was analyzed with multiple lasers simultaneously. FIG. 5A shows two-color (green-red) coincidence of dye-labeled antibodies recognizing the Akt1 C-terminus (Cy3, green) and PH domain (Cy5, red), indicating specific detection of Akt1. Both active and inactive Akt1 yield green-red coincident peaks; no such peaks were detected in the absence of Akt1 or in the presence of a GST control. In FIG. 5B, blue-red coincidence of dye-labeled antibodies recognizing Akt1 phosphorylated on Ser473 (Alexa488, blue) and the Akt1 PH domain (Cy5, red), performed simultaneously with that in panel A, indicates specific detection of active Akt1 molecules. Only active Akt1 yields blue-red coincident peaks, whereas no such peaks were detected in the presence of inactive Akt1, which is not phosphorylated on Ser473. FIG. 5C is a graph depicting the number of molecules versus percent active Akt1. Three-color coincidence of fluorophore labeled antibodies recognizing the Akt1 PH domain (Cy5, red), C-terminus (Cy3, green), and phosphorylated Ser473 (Alexa488, blue), indicating quantitation of the relative levels of active Akt1 are shown. Each sample contained 100 nM total Akt1 comprised of the indicated fraction of active Akt1 with the remainder being inactive Akt1. The number of molecules detected increased linearly with the proportion of active Akt1 molecules due to a fractional increase in the number of enzyme molecules phosphorylated on Ser473.

### Example 5

### Complex Analysis

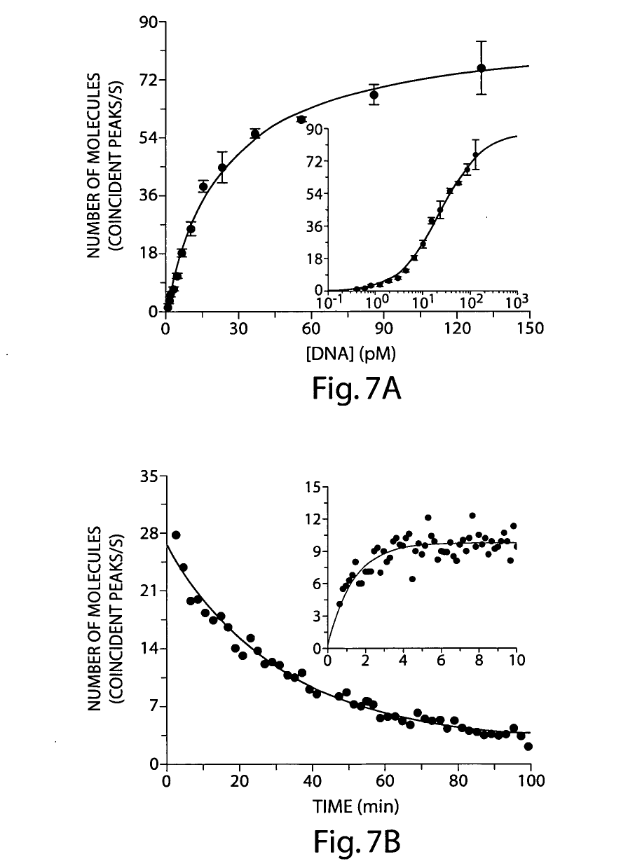

FIGS. 6A-6C demonstrate schematically the complexes to be formed between a nucleic acid and a zinc finger domain (ZFD). FIG. 7A shows the results of the binding reaction once equilibrium is reached. FIG. 7B illustrates the kinetic analysis of this system. ZFD of the Early Growth Response Protein Egr-1 binding to duplex DNA target was studied by mixing Bodipy-F1 labeled ZFD and Cy5 labeled duplex DNA target. Dissociation rate constant (k_{off}) of ZFD-DNA measured using single molecule analysis matched published data. Similarly, measured Kd (the dissociation constant which is equal to 1/Ka, where Ka is the equilibrium constant) matched published data.

### Equivalents

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description. Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

The phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

1. A method for detecting a protein comprising
contacting a sample with a first and a second protein-specific probe, and
detecting the binding of both the first and the second protein-specific probe to a single protein as coincident signals,
wherein the first and the second protein-specific probes are labeled with first and second detectable labels, respectively, that are distinguishable from each other, and
wherein the binding of both the first and the second protein-specific probes to a single protein indicates that the protein is present in the sample.

2. The method of claim 1, wherein the first and second protein-specific probes are antibodies or antibody fragments.

3. The method of claim 1, wherein the first and second protein-specific probes bind to different regions of the protein.

4. The method of claim 1, wherein the first and second detectable labels are fluorophores.

5. The method of claim 1, wherein the first and second detectable labels are Alexa488 and Cy5.

6. The method of claim 1, wherein the second protein-specific probe is specific for a protein modification.

7. The method of claim 1, further comprising detecting binding of the first and second protein-specific probes at various time points.

8. The method of claim 1, further comprising detecting binding of the first and second protein-specific probes in the presence of another molecule.

9. The method of claim 1, further comprising contacting the sample with a third probe protein-specific probe that is labeled with a third detectable label, and detecting the binding of the first, second and third protein-specific probes to the protein as coincident signals.

10. The method of claim 9, wherein the third detectable label is a fluorophore.

11. The method of claim 9, wherein the third detectable label is Cy3.

12. The method of claim 9, wherein the third protein-specific probe is specific for a protein modification.

13. The method of claim 6, wherein the protein modification is a phosphorylated amino acid.

14. The method of claim 9, further comprising detecting binding of the first, second and third protein-specific probes at various time points.

15. The method of claim 9, further comprising detecting binding of the first, second and third protein-specific probes in the presence of another molecule.

16. The method of claim 9, further comprising comparing the level of binding of the first and second protein-specific probes to the level of binding of the first, second and third protein-specific probes.

17-24. (canceled)

25. A method for detecting a microRNA (miRNA) comprising
contacting a sample with a first and a second miRNA-specific probe, and
detecting the binding of both the first and the second miRNA-specific probes to a single miRNA as coincident signals,
wherein the first and the second miRNA-specific probes are labeled with first and second detectable labels, respectively, that are distinguishable from each other, and
wherein the binding of both the first and the second miRNA-specific probes to a single miRNA indicates that the miRNA is present in the sample.

26-35. (canceled)

36. A method for detecting a complex comprising more than one component comprising
contacting a sample with a first component-specific probe and a second component-specific probe, and
detecting the binding of both the first component-specific probe and the second-component-specific probe to a single complex as coincident signals,
wherein the first component-specific probe and the second-component-specific probe are labeled with first and second detectable labels, respectively, that are distinguishable from each other, and
wherein the binding of both the first component-specific probe and the second-component-specific probe to a single complex indicates that the complex is present in the sample.

37-54. (canceled)

55. A method for detecting a complex comprising more than one component comprising
contacting a first component-specific probe labeled with a first detectable label to a sample comprising a second component labeled with a second detectable label that is distinguishable from the first detectable label, and
detecting binding of the first component-specific probe to a complex comprising the second component as coincident signals,
wherein coincident signals indicate that the complex is present in the sample.

56-77. (canceled)

78. A method for detecting a complex comprising more than one component comprising
contacting a first component labeled with a first detectable label with a second component labeled with a second detectable label that is distinguishable from the first detectable label, and
detecting binding of the first component to the second component as coincident signals,
wherein coincident signals indicate that the complex is present.

79-102. (canceled)
